(19) United States
(12) Patent Application Publication
(10) Pub. No.: US 2006/0120989 A1
(43) Pub. Date: Jun. 8, 2006

(54) Cosmetic composition comprising at least one polyol ether and at least one pasty substance

(76) Inventor: Audrey Ricard, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/274,309

(22) Filed: Nov. 16, 2005

Related U.S. Application Data
(60) Provisional application No. 60/629,316, filed on Nov. 19, 2004.

(30) Foreign Application Priority Data
Nov. 16, 2004 (FR) 04 52637

(51) Int. Cl. A61K 8/39

(52) U.S. Cl. 424/70.31

(57) ABSTRACT

The present disclosure relates to a composition comprising at least one fatty phase comprising at least one polyol ether and at least one pasty compound. The compositions as disclosed herein may be used as a care and/or make-up product for keratin materials, such as the skin, the lips and/or the superficial body growths.

This application claims benefit of U.S. Provisional Application No. 60/629,316, filed Nov. 19, 2004, the contents of which are incorporated herein by reference. This application also claims benefit of priority under 35 U.S.C. § 119 to French Patent Application No. 04 52637, filed Nov. 16, 2004, the contents of which are also incorporated herein by reference.

The present disclosure relates to a cosmetic composition comprising at least one fatty phase comprising at least one polyol ether. The composition may additionally comprise at least one other pasty compound. This composition may be used as a make-up and/or care composition for keratin materials such as the skin, the lips and the superficial body growths.

European Patent Application No. EP 1 306 074 describes a solid composition comprising a polyol ether, an oil and a wax of reversed polarity. This document describes how this combination makes it possible to obtain a solid composition that does not melt at a temperature of less than or equal to 37° C., while spreading easily on the skin to give a good deposit. The compositions may be lipstick compositions containing 1% of vitamin E.

The present disclosure relates to a cosmetic composition, which may be in the form of a stick or a paste. This composition can provide a deposit on the keratin materials which is comfortable, thick, intense in color and glossy.

The present disclosure further relates to a cosmetic composition comprising, in a physiologically acceptable medium, at least one fatty phase comprising at least one polyol ether chosen from polyalkylene glycol pentaerythritol ethers. The composition may comprise at least one pasty compound other than the at least one polyol ether.

This composition may be more glossy than the prior art compositions not comprising a polyol ether. In addition, the composition of the present disclosure may comprise a large amount of coloring matter, such as pigments, while allowing a homogeneous and comfortable mixture to be obtained.

The present disclosure also relates to an anhydrous composition comprising, in a physiologically acceptable medium, at least one fatty phase comprising at least one polyol ether, and at least one pasty compound.

The composition of the present disclosure may comprise, for example, a single fatty phase, and this fatty phase may be a continuous phase.

The compositions of the present disclosure may be anhydrous compositions, and may then comprise up to (i.e., less than or equal to) 10% by weight of a hydrophilic phase, relative to the total weight of the composition, for instance from 1 % to 5% by weight of hydrophilic phase, such as from 1% to 2% of hydrophilic phase, relative to the total weight of the composition. The hydrophilic phase may comprise water alone, or may comprise water and at least one additive chosen from hydrophilic and water-soluble additives such as polyols, gelling agents and/or active agents. If this hydrophilic phase is present, it may be, for example, dispersed in the fatty phase which forms a continuous phase.

According to one embodiment of the present disclosure, the composition is anhydrous, i.e., it contains only the fatty phase, or is essentially anhydrous, i.e., it comprises less than 5% by weight of water and/or hydrophilic or water-soluble additives.

Polyol Ether

In the composition according to the present disclosure, the at least one polyol ether can be chosen from, for example, from polyalkylene glycol pentaerythritol ethers, and sugar fatty alcohol ethers.

The at least one polyalkylene glycol pentaerythritol ether may comprise, for instance, from 1 to 450 oxyalkylenated units, such as from 1 to 200 oxyalkylenated units, for example from 1 to 100 oxyalkylenated units and from 1 to 50 oxyalkylenated units. For example, the at least one polyalkylene glycol pentaerythriotol may be chosen from polyethylene glycol pentaerythritol ethers comprising from 1 to 450 oxyethylenated units, such as from 1 to 200 oxyethylenated units, for instance from 1 to 100 oxyethylenated units and from 1 to 50 oxyethylenated units; and polypropylene glycol pentaerythritol ethers comprising from 1 to 450 oxypropylenated units, for instance from 1 to 200 oxypropylenated units, such as from 1 to 100 oxypropylenated units and from 1 to 50 oxypropylenated units. According to one embodiment of the present disclosure, the at least one polyalkylene glycol pentaerythriotol is chosen from polyethylene glycol pentaerythrityl ethers comprising 5 oxyethylenated units (5 EO) (CTFA name: PEG-5 Pentaerythrityl Ether), and polypropylene glycol pentaerythritol ethers comprising 5 oxypropylenated units (5 PO) (CTFA name: PPG-5 Pentaerythrityl Ether). By way of non-limiting example, the PEG-5 Pentaerythrityl Ether, PPG-5 Pentaerythrityl Ether and soya bean oil mixture marketed under the name “Lanolide” by the company Vevy, a mixture in which the constituents are in a 46/46/8 weight ratio: 46% of PEG-5 Pentaerythrityl Ether, 46% of PPG-5 Pentaerythrityl Ether and 85% of soya bean oil, may also be used.

The at least one sugar fatty alcohol ether may be chosen from C₈-C₂₂ fatty alcohol ethers; mixtures of C₈-C₂₂ fatty alcohol ethers of glucose, maltose, sucrose and fructose; C₁₄-C₂₂ fatty alcohol ethers; and mixtures of C₁₄-C₂₂ fatty alcohol ethers of methylglucose.

The C₈-C₂₂ and C₁₄-C₂₂ fatty alcohols forming the fatty unit of the sugar ethers comprise a saturated or unsaturated linear alkyl chain comprising from 8 to 22, or from 14 to 22, carbon atoms respectively. The fatty unit of the ethers obtained from the fatty alcohol may be chosen from, for example, the decyl, cetyl, behenyl, arachidyl, stearyl, palmityl, myristyl, lauryl, capryl and hexadecanyl units, and mixtures thereof, such as cetearyl (mixture of cetyl and stearyl).

By way of examples of sugar fatty alcohol ethers, non-limiting mention may be made of alkyl polyglucosides (APG) such as decyl glucoside and lauryl glucoside marketed for example by the company Henkel under the respective names PLANTAREN 2000 and PLANTAREN 1200; and cetearyl glucoside optionally as a mixture with cetostearyl alcohol, the mixture being marketed, for example, under the name MONTANOV 68 by the company Seppic (mixture in which the constituents are in a 12/46/42 ratio: 12% of cetearyl glucoside, 46% of cetyl alcohol and 42% of stearyl alcohol). As cetearyl glucoside that may be used, non-limiting mention may also be made of the products marketed under the name Tegocare CG90 by the company Goldschmidt and under the name EMULGADE KE3302 by the company Henkel. As APG that may be used, non-limiting mention may also be made of arachidyl glucoside, for example in the form of the mixture of arachidyl and behenyl alcohols and arachidyl glucoside, marketed under the name MONTANOV 202 by the company Seppic. Non-limiting mention may also be made of the branched or unsaturated chain alkyl polyglucosides such as isostearyl glucoside optionally as a mixture with isostearyl alcohol, marketed for example under the name MONTANOV W013 by the company Seppic, and oleyl glucoside optionally as a mixture with oleyl alcohol, marketed by the company Seppic. It is also possible to use a mixture of these alkyl polyglucosides. According to one embodiment of the present disclosure, the mixture of the alkyl polyglucoside as defined above with the corresponding fatty alcohol may be in the form of a self-emulsifiable composition as described, for example, in the PCT International Application Publication No. WO-A-92/06778.

The amount of the at least one polyol ether in the compositions of the disclosure can vary widely, and can range for example from 0.5% to 40% by weight, for instance from 1 % to 30% by weight, such as from 50% to 25% by weight, relative to the total weight of the composition.

Pasty Substance

The composition according to the present disclosure can additionally comprise at least one other pasty compound different from the compound described above.

As used herein, the expression “pasty,” is understood to mean a lipophilic fatty compound with a reversible solid/liquid change of state having, in the solid state, an anisotropic crystalline organization, and having, at the temperature of 23° C., a liquid fraction and a solid fraction. In at least one embodiment, the expression “pasty” may be understood to mean polyvinyl laurate.

The at least one additional pasty compound, for the purposes of the present disclosure, may have a hardness, at 20° C., ranging from 0.001 MPa to 0.5 MPa, such as from 0.002 MPa to 0.4 MPa.

The hardness is measured according a method of penetration of a probe into a sample of compound and, for instance, with the aid of a texture analyzer (for example the TA-XT2i from Rheo) equipped with a stainless steel spindle 2 mm in diameter. The measurement of hardness is performed at 20° C. at the center of 5 samples. The spindle is introduced into each sample at a pre-speed of 1 mm/s and then at a measuring speed of 0.1 mm/s, the penetration depth being 0.3 mm. The hardness value measured is that of the maximum peak.

This pasty compound may, at the temperature of 23° C., be in the form of a liquid fraction and of a solid fraction. In other words, the onset of melting temperature of the pasty compound may be less than 23° C. The liquid fraction of the pasty compound, measured at 23° C., comprises from 9% to 97% by weight, relative to the total weight of the compound. This liquid fraction at 23° C., can comprise for example, from 15% to 85%, such as from 40% to 85% by weight, relative to the total weight of the compound.

The liquid fraction by weight of the pasty compound at 23° C. is equal to the ratio of the heat of fusion consumed at 23° C. to the heat of fusion of the pasty compound.

The heat of fusion of the pasty compound is the heat consumed by the compound to change from the solid state to the liquid state. The pasty compound is said to be in the solid state when all of its mass is in the solid crystalline form. The pasty compound is said to be in the liquid state when all of its mass is in the liquid form.

The heat of fusion of the pasty compound is equal to the area under the curve of the thermogram obtained with the aid of a differential scanning calorimeter (D.S.C.), such as the calorimeter sold under the name MDSC 2920 by the company TA Instruments, with a temperature rise of 5° C. or 10° C. per minute, according to the ISO 11357-3:1999 standard. The heat of fusion of the pasty compound is the amount of energy required to make the compound change from the solid state to the liquid state. It is expressed in J/g.

Thus, the heat of fusion consumed at 23° C. is the amount of energy absorbed by the sample to change from the solid state to the state that it has at 23° C., comprising a liquid fraction and a solid fraction.

The liquid fraction of the pasty compound measured at 32° C., can, for example, comprise from 30% to 100% by weight, relative to the total weight of the compound, for instance, from 80% to 100%, such as from 90% to 100% by weight, relative to the total weight of the compound. When the liquid fraction of the pasty compound, measured at 32° C., is equal to 100%, the temperature at the end of the melting range of the pasty compound is less than or equal to 32° C.

The liquid fraction of the pasty compound, measured at 32° C., is equal to the ratio of the heat of fusion consumed at 32° C. to the heat of fusion of the pasty compound. The heat of fusion consumed at 32° C. is calculated in the same manner as the heat of fusion consumed at 23° C.

The at least one pasty compound can be chosen from, for example, synthetic compounds and compounds of plant origin. The at least one pasty compound may also be obtained by synthesis from starting materials of plant origin.

In at least one embodiment, the at least one pasty compound can be chosen from, by way of non-limiting example:

lanolin and its derivatives;

polymeric and non-polymeric silicone compounds;

polymeric and non-polymeric fluorinated compounds;

vinyl polymers, such as:
homopolymers of olefins,
copolymers of olefins,
homopolymers and copolymers of hydrogenated dienes,
linear or branched oligomers, or homo- or copolymers of alkyl (meth)acrylates, for instance, comprising a C₈-C₃₀ alkyl group,
oligomers, homo- and copolymers of vinyl esters having C₈-C₃₀ alkyl groups, and
oligomers, homo- and copolymers of vinyl ethers having C₈-C₃₀ alkyl groups;

fat-soluble polyethers resulting from polyetherification of at least one C₂-C₁₀₀, such as C₂-C₅₀, diol, and

esters.

The at least one pasty compound can be, for example, polymeric, such as hydrocarbon-based.

The at least one pasty compound can also be a silicone and fluorinated pasty compound, such as polymethyl trifluoropropyl methylalkyl dimethylsiloxane, manufactured under the name X22-1088 by SHIN ETSU.

When the at least one pasty compound is a silicone and/or fluorinated polymer, the composition can comprise, for example, a compatibilizing agent, for instance short-chain esters such as isodecyl neopentanoate.

Among the fat-soluble polyethers that may be used, non-limiting mention may be made of copolymers of ethylene oxide and/or propylene oxide with C₆-C₃₀ long-chain alkylene oxides, for example, chosen such that the weight ratio of the ethylene oxide and/or propylene oxide to alkylene oxides in the copolymer ranges from 5:95 to 70:30. In this family, further non-limiting mention may be made of copolymers such that the long-chain alkylene oxides are arranged in blocks having an average molecular weight ranging from 1,000 to 10,000, for example a polyoxyethylene/polydodecyl glycol block copolymer such as the ethers of dodecanediol (22 mol) and of polyethylene glycol (45 EO) marketed under the trade mark ELFACOS ST9 by Akzo Nobel.

Among the esters that may be used, in at least one embodiment non-limiting mention may be made of:

esters of an oligomeric glycerol, such as diglycerol esters, for instance the condensates of adipic acid and glycerol, for which a portion of the hydroxyl groups of the glycerols have reacted with a mixture of fatty acids such as stearic acid, capric acid, stearic acid and isostearic acid, and 12-hydroxystearic acid, such as, for example, those marketed under the trade mark SOFTISAN 649 by the company Sasol,

arachidyl propionate marketed under the trade mark WAXENOL 801 by Alzo,

phytosterol esters,

triglycerides of fatty acids and derivatives thereof,

pentaerythritol esters,

non-crosslinked polyesters resulting from polycondensation of a dicarboxylic acid or a linear or branched C₄-C₅₀ polycarboxylic acid with a diol or a C₂-C₅₀ polyol,

aliphatic esters of an ester resulting from the esterification of an aliphatic hydroxycarboxylic acid ester with an aliphatic carboxylic acid.

The aliphatic carboxylic acids that may be used comprise from 4 to 30, such as from 8 to 30 carbon atoms. The at least one aliphatic carboxylic acids can be chosen from, for instance, hexanoic acid, heptanoic acid, octanoic acid, 2-ethylhexanoic acid, nonanoic acid, decanoic acid, undecanoic acid, dodecanoic acid, tridecanoic acid, tetradecanoic acid, pentadecanoic acid, hexadecanoic acid, hexyldecanoic acid, heptadecanoic acid, octadecanoic acid, isostearic acid, nonadecanoic acid, eicasanoic acid, isoarachidic acid, octyidodecanoic acid, heneicosanoic acid, and docosanoic acid.

In at least one embodiment of the present disclosure, the at least one aliphatic carboxylic acid is branched.

The at least one aliphatic hydroxycarboxylic acid ester can be obtained from, for example, a hydroxylated aliphatic carboxylic acid comprising from 2 to 40 carbon atoms, for instance from 10 to 34 carbon atoms, such as from 12 to 28 carbon atoms; and from 1 to 20 hydroxyl groups, for example from 1 to 10 hydroxyl groups, such as from 1 to 6 hydroxyl groups.

By way of non-limiting example, the at least one aliphatic hydroxycarboxylic acid ester can be chosen from:
a) partial and total esters of saturated linear monohydroxylated aliphatic monocarboxylic acids;
b) partial and total esters of unsaturated monohydroxylated aliphatic monocarboxylic acids;
c) partial and total esters of saturated monohydroxylated aliphatic polycarboxylic acids;
d) partial and total esters of saturated polyhydroxylated aliphatic polycarboxylic acids; and
e) partial and total esters of C₂ to C₁₀ aliphatic polyols which have reacted with a mono- or polyhydroxylated aliphatic mono- or polycarboxylic acid.

The at least one aliphatic ester of an ester can be chosen from, in at least one embodiment:
esters resulting from the esterification reaction of hydrogenated castor oil with isostearic acid in a 1 to 1 (1/1) ratio or monoisostearate of hydrogenated castor oil, esters resulting from the esterification reaction of hydrogenated castor oil with isostearic acid in a 1 to 2 (1/2) ratio or diisostearate of hydrogenated castor oil, and
esters resulting from the esterification reaction of hydrogenated castor oil with isostearic acid in a 1 to 3 (1/3) ratio or triisostearate of hydrogenated castor oil.

The at least one pasty compound can be present in the presently disclosed composition(s) in an amount ranging from 1% to 99%, for instance from 1% to 60%, such as from 2% to 30% and from 5% to 15% by weight, relative to the total weight of the composition.

According to one embodiment of the present disclosure, the cosmetic composition as disclosed herein comprises, in a physiological acceptable medium, at least one fatty phase comprising at least one polyol ether chosen from polyalkylene glycol pentaerythritol ethers, and at least one copolymer of vinylpyrrolidone

Thus, the composition according to the present disclosure may also comprise at least one polyalkylene chosen from copolymers of vinylpyrrolidone (VP), such as copolymers of vinylpyrrolidone and C₂ to C₄₀, for instance C₃ to C₂₀, alkenes. By way of non-limiting example of a copolymer of VP which can be used herein, there may be mentioned copolymers of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene, VP/acrylic acid/lauryl methacrylate, and mixtures thereof.

The at least one vinylpyrrolidone copolymer can have a melting point ranging from 30° C. to 40° C. The at least one vinylpyrrolidone copolymer can be present in an amount ranging from 0.1% to 10% by weight, relative to the total weight of the composition, for example from 0.5% to 5% by weight, relative to the total weight of the composition.

Oil

The at least one fatty phase of the composition according to the present disclosure comprises at least one oil. As used herein, the expression “oil” is understood to mean any physiologically acceptable non-aqueous medium which is liquid at room temperature (25° C.) and atmospheric pressure (760 mmHg).

The at least one oil can be present in an amount ranging, for example, from 20% to 80% by weight, such as from 30% to 70% by weight, relative to the total weight of the composition.

The at least one oil can be chosen from hydrocarbon and/or silicone and/or fluorinated oils. The at least one oil can be of animal, plant, mineral or synthetic origin. As used herein, the expression “hydrocarbon oil” is understood to mean any oil predominantly comprising carbon and hydrogen atoms, and optionally at least one group chosen from ester, ether, fluorinated, carboxylic acid and/or alcohol groups. In addition, the at least one oil used may be chosen from volatile and/or non-volatile oils. As used herein, the expression “volatile oil” is understood to mean an oil capable of evaporating at room temperature from a support onto which it is applied, i.e., an oil having a vapor pressure which is measurable at 25° C. and 1 atmosphere, for example greater than 0 Pa, for instance ranging from 10⁻³ mmHg to 300 mmHg (0.13 Pa to 40,000 Pa). There may be mentioned, by way of non-limiting example, among the volatile oils that may be used, volatile silicone oils such as cyclic or linear volatile silicones. Non-limiting mention may also be made of volatile hydrocarbon oils such as isoparaffins and fluorinated volatile oils.

The at least one oil which may be used in the composition of the present disclosure can be chosen from polar and apolar, i.e., non-polar, oils.

The polar oils which may be used comprise in their chemical structure at least one non-ionic polar group, and for instance at least two non-ionic or ionic polar groups such as the following groups:
COOH;
OH which is mono- or di-substituted (primary or secondary);
PO₄;
NHF;
NR₁R₂, wherein R₁ and R₂, which may be identical or different, are chosen from linear and branched C, to C₂₀ alkyl and alkoxy radicals; R₁ and R₂ may optionally form a ring; and
groups of formula: 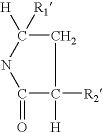
   wherein R₁′ and R₂′, which may be identical or different, are chosen from hydrogen atoms and linear and branched C₁ to C₂₀ alkyl and alkoxy chains.

The polarity may be described by the Hansen solubility parameter δₐ. This parameter characterizes, for a given constituent, the energy corresponding to the polar interactions (δₚ) and the hydrogen-bond-type interactions (δₕ) existing between the molecules of the constituent.
δₐ=\sqrt{δₚ²+δₕ²}

The apolar oils have a δₐ value equal to 0. In at least one embodiment, the apolar oils according to the present disclosure may be chosen from:
silicone oils, such as linear or cyclic, volatile or non-volatile polydimethylsiloxanes (PDMS) which are liquid at room temperature; phenylated silicones such as phenyltrimethicones, phenlydimethicones, phenyltrimethylsiloxydiphenylsiloxanes, diphenyidimethicones, diphenylmethyldiphenyltrisiloxanes, and 2-phenylethyl trimethylsiloxysilicates;
linear or branched hydrocarbons of synthetic or inorganic origin such as volatile or non-volatile paraffin oils and derivatives thereof; liquid paraffin; liquid lanolin; polydecenes; hydrogenated polyisobutene such as Parleam® oil; squalane; hydrogenated isoparaffin; isohexadecane; isododecane; and mixtures thereof.

The polar oils have a δₐ value different from 0, i.e., greater than 0. By way of non-limiting example, the polar oils used in the composition of the present disclosure can be chosen from:
oils of plant origin, hydrocarbon oils with a high content of triglycerides comprising fatty acid esters of glycerol in which the fatty acids may have varying chain lengths from C₄ to C₂₀, it being possible for the fatty acids to be linear or branched, saturated or unsaturated. Among the oils of plant origin, non-limiting mention may be made of jojoba, wheat germ, maize, sunflower, shea butter, castor, sweet almond, macadamia, apricot, soya bean, cottonseed, lucerne, poppyseed, pumpkinseed, sesame, gourd, rapeseed, avocado, hazelnut, grapeseed, blackcurrant seed, evening primrose, millet, barley, quinoa, olive, rye, safflower, candlenut, passionflower, rose-musk and coriander oils; or alternatively, non-limiting mention may also be made of caprylic/capric acid triglycerides such as those sold by the company Stearineries Dubois or those sold under the names MIGLYOL 810, 812 and 818 by the company Dynamit Nobel;
synthetic oils or synthetic esters of formula R₅COOR₆, in which R₅ is chosen from the residues of linear and branched fatty acids comprising from 1 to 40 carbon atoms and R₆ is chosen from hydrocarbon chains, such as branched hydrocarbon chains, comprising from 1 to 40 carbon atoms, provided that R₅+R₆ is ≧10 carbon atoms, such as, for example Purcellin oil (cetostearyl octanoate), isononyl isononanoate, C₁₂ to C₁₅ alcohol benzoate, isoprypyl myristate, 2-ethylhexyle palmitate, isostearyl isostearate, isoprypyl isostearate, alcohol or polyalcohol octanoates, decanoates or ricinoleates; hydroxylated esters such as isostearyl lactate, diisostearyl malate; C₁₂-C₁₅ alkyl benzoate; and pentaerythritol esters;
synthetic ethers comprising from 10 to 40 carbon atoms;
C₈ to C₂₆ fatty alcohols, such as oleyl alcohol, isostearyl alcohol and octyldodecanol; and
mixtures thereof.
   Wax

The at least one fatty phase of the composition according to the present disclosure may further comprise at least one wax. As used herein, the term “wax,” is understood to mean a lipophilic fatty compound which is solid at room temperature (25° C.), with a reversible solid/liquid change of state, having a melting point ranging from 40° C. to 200° C., and having an anisotropic crystalline organization in the solid state.

Any type of wax may be used. Thus, by way of non-limiting example, the at least one wax may be chosen from waxes of natural origin, such as plant or animal origin, waxes of mineral origin, and waxes of synthetic origin. Among the waxes which may be used in the composition of the present disclosure, further non-limiting mention may be made of, for example, bees wax, Montan wax, Carnauba wax, Candelilla wax, Chinese wax, linseed wax, pine wax, cotton wax, Ouricoury wax, lignite wax, rice bran wax, sugar cane wax, Japan wax, cork fibre wax, paraffin waxes, microcrystalline waxes, lanolin wax, ozokerites, hydrogenated oils having a melting point greater than 40° C., such as hydrogenated jojoba oil, polyethylene waxes which are derived from the polymerization of ethylene, waxes obtained by Fischer-Tropsch synthesis, fatty acid esters and glycerides which are solid at 40° C., silicone waxes such as alkyl, alkoxy and/or esters of poly(di)methylsiloxane which are solid at 40° C., and mixtures thereof.

As discussed above, when the composition as disclosed herein comprises at least one polar oil, it should comprise at least one apolar wax. When the composition comprises at least one apolar oil, it should comprise at least one polar wax. When the composition comprises at least one polar oil and at least one apolar oil, it should comprise at least one apolar wax.

As discussed above for the oils, the polarity may be described by the Hansen solubility parameter δₐ according to the equation indicated above.

The so-called apolar waxes have a δₐ value equal to 0. They can be, in at least one embodiment, hydrocarbon waxes comprising mainly carbon and hydrogen atoms, or silicone waxes. Among the hydrocarbon waxes that may be used, non-limiting mention may be made of microcrystalline waxes, oxokerite, paraffin waxes, and (unmodified) polyethylene waxes.

The so-called polar waxes are waxes comprising polar groups as indicated above for the oils, and they have a δₐ value greater than 0. By way of non-limiting example, the polar waxes can be chosen from waxes of animal origin, waxes of plant origin, waxes of synthetic origin comprising polar groups and silicone waxes comprising polar groups. Further non-limiting mention may be made, for example, of Montan wax, Carnauba wax, Candelilla wax, Chinese wax, linseed wax, pine wax, cotton wax, Ouricoury wax, lignite wax, rice bran wax, sugar cane wax, Japan wax, cork fibre wax, polyglycerolated bees waxes, hydrogenated oils, fatty acid esters and glycerides which are solid at 40° C., and silicone waxes comprising at least one ester group.

The at least one wax can be present, in a total amount (polar and/or apolar), ranging, for example, from 5% to 40% by weight, such as from 10% to 30% by weight, relative to the total weight of the composition.

Hydrophilic Phase

The composition of the present disclosure may comprise, in an amount ranging from 0% to 10% by weight, relative to the total weight of the composition, at least one hydrophilic phase, for instance, from 1% to 5% by weight, which may comprise water and/or at least one hydrophilic or water-soluble additive (such as active agents and/or gelling agents). For example, it may comprise moisturizers such as glycerine. The at least one hydrophilic constituent optionally present can be, for example, also dispersed in the at least one fatty phase comprising the oils and waxes.

Particulate Phase

The composition according to the present disclosure may additionally comprise at least one particulate phase. The at least one particulate phase can be present in an amount ranging, for example, from 0% to 30% by weight, for instance, from 0% to 20% by weight, relative to the total weight of the composition. When at least one particulate phase is present, it can generally be present in an amount ranging from at least 0.05% by weight, relative to the total weight of the composition. The at least one particulate phase can be present in an amount ranging, for example, from 0.05% to 30% by weight, such as from 1% to 20% by weight, relative to the total weight of the composition.

The at least one particulate phase can comprise particles chosen from pigments, pearlescent agents, and fillers. These pigments, pearlescent agents and fillers are chosen from those customarily used in cosmetic compositions. As used herein, the expression “pigments” is understood to mean white or colored, inorganic or organic particles intended to color and/or opacify the composition. As used herein, the expression “fillers” is understood to mean colorless or white, inorganic or synthetic, lamellar or non-lamellar particles intended to give body or firmness to the composition, and/or smoothness, mattness and uniformity to the composition. As used herein, the expression “pearlescent agents” is understood to mean iridescent particles which reflect light.

The pigments that can be used as disclosed herein can be white and/or colored, inorganic and/or organic, and of micrometer or nanometer size. There may be mentioned, by way of non-limiting example, among the inorganic pigments that may be used, titanium, zirconium or cerium dioxides, and zinc, iron or chromium oxides, and ferric blue. Among the organic pigments that may be used, non-limiting mention may be made of carbon black, and barium, strontium, calcium and aluminium lacquers.

Among the pearlescent agents which can be used in the composition of the present disclosure, non-limiting mention may be made of, for example, mica coated with titanium oxide, with iron oxide, with natural pigment or with bismuth oxychloride, and coloured mica-titanium.

The fillers may be inorganic or synthetic, lamellar or non-lamellar. As fillers which may be used in the composition of the present disclosure, non-limiting mention may be made of, for example, talc, mica, silica, kaolin, Nylon powders, polyethylene powders, Teflon®, modified or unmodified starch, mica-titanium, natural pearl, boron nitride, microspheres such as EXPANCEL (Nobel Industrie), POLYTRAP (Dow Corning), lauroyl lysine, and microbeads of silicone resin (TOSPEARLS from Toshiba, for example).

For example, the composition according to the present disclosure can comprise at least one coloring material which may be chosen from the lipophilic colorants or hydrophilic colorants normally used in cosmetic or dermatological compositions, and from the pigments and pearlescent agents described above, and mixtures thereof. The at least one coloring material can be present in an amount ranging from 0.01% to 40% by weight, such as from 5% to 25% by weight, relative to the total weight of the composition.

Adjuvants

The composition according to the present disclosure may additionally comprise at least one adjuvant customarily used in the field considered, such as the cosmetic field, for instance antioxidants; perfumes; essential oils; preservatives; cosmetic active agents; vitamins such as vitamin E (tocopherol) and its derivatives (for example acetate), vitamin A (retinol) and its derivatives (for example retinyl palmitate), vitamin C (ascorbic acid) and its derivatives (for example ascorbyl palmitate), the derivatives of these vitamins being, for example, esters including the palmitate and acetate; essential fatty acids; sphingolipids and ceramides; self-tanning compositions such as DHA (dihydroxyacetone); sun-screening agents such as for example octylmethoxycinnamate (PARSOL MCX), 3-benzophenone (UVINUL M40), butylmethoxydibenzoylmethane (PARSOL 1789); surfactants; polymers; and mixtures thereof. The at least one adjuvant may be present in the composition in an amount ranging from 0% to 20% by weight relative to the total weight of the composition.

Of course, persons skilled in the art will be careful to choose any of these optional additional compound(s), and/or the quantity thereof, such that the beneficial properties of the composition according to the present disclosure are not, or not substantially, impaired by the additions envisaged.

The methods for manufacturing the compositions according to the present disclosure are in no way different from the methods which are conventionally used in the cosmetic field and which are entirely known to persons skilled in the art.

The compositions according to the present disclosure may, for example, constitute a care product and/or a make-up product for keratin materials, such as the skin, the lips and the superficial body growths such as the nails, the eyelashes, the eyebrows and the hair. The make-up products may be colored and may comprise pigments. When in the form of make-up products, the compositions of the present disclosure may be, for example, in a form chosen from a foundation, a lipstick, a blusher, an eye shadow, a mascara or an eye liner.

For further example, the product may be in the form of a cast powder, a product in a dish (foundation, blusher, eye shadow), or a product in the form of a stick (lipstick or lip care stick). According to one embodiment of the present disclosure, the composition is in the form of a stick, for instance for lip care or for lip make-up, such as lipstick.

Accordingly, the present disclosure also relates to the cosmetic use of a cosmetic composition as defined above, for the care and/or treatment of the skin, the lips and/or the superficial body growths, and in one embodiment, for moisturizing the lips.

The present disclosure also relates to the cosmetic use of a cosmetic composition as defined above, for the making up of the skin, the lips and/or the superficial body growths.

The present disclosure further relates to a cosmetic method for treating chapped and/or dry lips, comprising applying to the lips a cosmetic composition as defined above.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements.

The present disclosure is illustrated in greater detail by the following non-limiting examples, in which the percentages are given by weight, unless otherwise stated.

### EXAMPLES

| | |
|---|---|
| Example 1: Lipstick | |
| Lanolide ® (company Vevy) | 15% |
| Diisostearyl malate | 10% |
| Polydimethylsiloxane (5 cSt) | 5% |
| Pentaerythrityl Tetraisostearate | 10% |
| Tridecyl Trimellitate | 10% |
| 2-Decyltetradecanoic acid triglyceride | 3% |
| Isononyl Isononanoate | 10% |
| Plant isostearic and adipic fatty acid | 6% |
| esters (SOFTISAN 100) | |
| Polybutene | 5% |
| Polyethylene wax | 6.3% |
| Microcrystalline wax | 4% |
| Pigments and colorants | 11% |
| Vinylpyrrolidone/eicosene copolymer | 2% |
| DL-bisabolol | 0.3% |
| N-lauroyl lysine | 2.4% |
| Preservative | qs |
| Example 2: Lipstick | |
| Lanolide ® (company Vevy) | 18.6% |
| Isostearyl neopentanoate | 13.3 |
| Bisglyceryl polyadipate (SOFTISAN 100) | 3.35 |
| Phenyl trimethicone | 7.6 |
| Phenyl trimethicone 20 cSt | 3.6 |
| Bentone | 1.44 |
| Isopropyl isostearate | 22.56 |
| Ozokerite (mineral wax) | 1.75 |
| Polyethylene wax | 6.5 |
| Polyethylene wax | 3.2 |
| Titanium dioxide | 0.73 |
| Red 7 | 3.4 |
| Yellow 6 lake | 6.12 |
| Kaolin | 5 |
| N-lauroyl lysine | 2.5 |
| DL-bisabolol | 0.3 |
| BHT | qs |

Procedure:

The pigments were ground in a three roll mill with the oily phase and the gel phase. The gel was obtained with a turbine until swelling and homogenization of the bentone was obtained. The waxes were weighed in a jacketed saucepan with circulation of oil, and then heated with stirring (turbine). After complete melting, the ground material, the phenyl trimethicones and the active agents were added. After homogenization of the mixture, the juice was poured at 100° C. into a siliconized alu mould at 42° C. After recrystallization of the sticks at −4° C. for at least 30 min, they were introduced into the “ACs” and then left to stabilize for 24 h at 20° C. before any evaluation.

What is claimed is:

1. A cosmetic composition comprising, in a physiologically acceptable medium, at least one fatty phase comprising
(i) at least one polyol ether chosen from polyalkylene glycol pentaerythritol ethers, present in an amount ranging from 0.5% to 40% by weight, relative to the total weight of the composition, and
(ii) at least one pasty compound other than the at least one polyol ether, present in an amount ranging from 2% to 30% by weight, relative to the total weight of the composition.

2. The cosmetic composition according to claim 1, wherein the at least one polyol ether is chosen from polyethylene glycol pentaerythritol ethers comprising from 1 to 450 oxyethylenated units, and polypropylene glycol pentaerythritol ethers comprising from 1 to 450 oxypropylenated units.

3. The cosmetic composition according to claim 2, wherein the at least one polyol ether is chosen from polyethylene glycol pentaerythritol ethers comprising 5 oxyethylenated units, and polypropylene glycol pentaerythritol ethers comprising 5 oxypropylenated units.

4. The cosmetic composition according to claim 3, wherein the at least one polyol ether is in the form of a polyethylene glycol pentaerythritol ether comprising 5 oxyethylenated units/polypropylene glycol pentaerythritol ether comprising 5 oxypropylenated units/soya bean oil mixture.

5. The cosmetic composition according to claim 1, wherein the at least one polyol ether is present in an amount ranging from 1 % to 30% by weight, relative to the total weight of the composition.

6. The cosmetic composition according to claim 1, wherein the at least one pasty compound has a hardness, at 20° C., ranging from 0.001 MPa to 0.5 MPa.

7. The cosmetic composition according to claim 6, wherein the at least one pasty compound has a hardness, at 20° C., ranging from 0.002 MPa to 0.4 MPa.

8. The cosmetic composition according to claim 1, wherein the at least one pasty compound comprises a liquid fraction at 23° C. present in an amount ranging from 9% to 97% by weight, relative to the total weight of the at least one pasty compound.

9. The cosmetic composition according to claim 8, wherein the at least one pasty compound comprises a liquid fraction at 23° C. present in an amount ranging from 40% to 85% by weight, relative to the total weight of the at least one pasty compound.

10. The cosmetic composition according to claim 1, wherein the at least one pasty compound comprises a liquid fraction at 32° C. present in an amount ranging from 30% to 100% by weight, relative to the total weight of the at least one pasty compound.

11. The cosmetic composition according to claim 10, wherein the at least one pasty compound comprises a liquid fraction at 32° C. present in an amount ranging from 90% to 100% by weight, relative to the total weight of the at least one pasty compound.

12. The cosmetic composition according to claim 1, wherein the at least pasty compound is chosen from
polymeric and non-polymeric silicone compounds,
polymeric and non-polymeric fluorinated compounds,
vinyl polymers,
fat-soluble polyethers resulting from polyetherification of at least one C₂-C₁₀₀ diol, and
esters.

13. The cosmetic composition according to claim 12, wherein the vinyl polymers are chosen from:
homopolymers of olefins,
copolymers of olefins,
homopolymers and copolymers of hydrogenated dienes,
linear or branched oligomers, or homo- or copolymers of alkyl (meth)acrylates, optionally having a C₈-C₃₀ alkyl group,
oligomers, homo- and copolymers of vinyl esters having C₈-C₃₀ alkyl groups, and
oligomers, homo- and copolymers of vinyl ethers having C₈-C₃₀ alkyl groups.

14. The at least one cosmetic composition according to claim 1, wherein the at least one pasty compound is hydrocarbon-based.

15. The cosmetic composition according to claim 12, wherein the at least one pasty compound is polymethyl trifluoropropyl methylalkyl dimethylsiloxane.

16. The cosmetic composition according to claim 12, wherein the at least one fat-soluble polyether is chosen from copolymers of ethylene oxide and/or propylene oxide with C₆-C₃₀ long-chain alkylene oxides.

17. The cosmetic composition according to claim 16, wherein the weight ratio of the ethylene oxide and/or propylene oxide to the alkylene oxides in the copolymer ranges from 5:95 to 70:30.

18. The cosmetic composition according to claim 13, wherein the at least one fat-soluble polyether is a polyoxyethylene/polydodecyl glycol block copolymer.

19. The cosmetic composition according to claim 13, wherein the at least one ester is chosen from
esters of an oligomeric glycerol for which a portion of the hydroxyl groups of the glycerol have reacted with a mixture of fatty acids,
arachidyl propionate,
phytosterol esters,
triglycerides of fatty acids and derivatives thereof,
pentaerythritol esters,
non-crosslinked polyesters resulting from polycondensation between a dicarboxylic acid or a linear or branched C₄-C₅₀ polycarboxylic acid and a diol or a C₂-C₅₀ polyol, and
aliphatic esters of an ester resulting from the esterification of an aliphatic hydroxycarboxylic acid ester with an aliphatic carboxylic acid.

20. A cosmetic composition comprising, in a physiologically acceptable medium, at least one fatty phase comprising at least one polyol ether chosen from polyalkylene glycol pentaerythritol ethers, and
at least one pasty compound which is a copolymer of vinylpyrrolidone (VP).

21. The cosmetic composition according to claim 20, wherein the at least one vinylpyrrolidone copolymer is chosen from copolymers of vinylpyrrolidone and a C₂ to C₄₀ alkene.

22. The cosmetic composition according to claim 21, wherein the at least one vinylpyrrolidone copolymer is chosen from copolymers of vinylpyrrolidone and a C₃ to C₂₀ alkene

23. The cosmetic composition according to claim 20, wherein the at least one vinylpyrrolidone copolymer is chosen from the copolymers of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene, and VP/acrylic acid/lauryl methacrylate.

24. The cosmetic composition according to claim 1, further comprising at least one oil in an amount ranging from 20% to 80% by weight, relative to the total weight of the composition.

25. The cosmetic composition according to claim 24, further comprising at least one oil in an amount ranging from 30% to 70% by weight, relative to the total weight of the composition.

26. The cosmetic composition according to claim 1, further comprising at least one polar oil chosen from oils of plant origin; synthetic oils and synthetic esters of formula R₅COOR₆ in which R₅ is chosen from the residues of linear and branched fatty acids comprising from 1 to 40 carbon atoms and R₆ is chosen from hydrocarbon chains comprising from 1 to 40 carbon atoms, provided that R₅+R₆ is ≧10; synthetic ethers comprising from 10 40 carbon atoms; and C₈ to C₂₆ fatty alcohols; and mixtures thereof.

27. The cosmetic composition according to claim 1, further comprising at least one wax chosen from waxes of natural origin; waxes of mineral origin; and waxes of synthetic origin.

28. The cosmetic composition according to claim 27, wherein the at least one wax is chosen from bees wax, Montan wax, Carnauba wax, Candelilla wax, Chinese wax, linseed wax, pine wax, cotton wax, Ouricoury wax, lignite wax, rice bran wax, sugar cane wax, Japan wax, cork fibre wax, paraffin waxes, microcrystalline waxes, lanolin wax, ozokerites, hydrogenated oils having a melting point greater than 40° C., polyethylene waxes, waxes obtained by Fischer-Tropsch synthesis, fatty acid esters and glycerides which are solid at 40° C., and silicone waxes.

29. The cosmetic composition according to claim 27, wherein the at least one wax is present in an amount ranging from 5% to 40% by weight, relative to the total weight of the composition.

30. The cosmetic composition according to claim 29, wherein the at least one wax is present in an amount ranging from 10% to 30% by weight, relative to the total weight of the composition.

31. The cosmetic composition according to claim 1, further comprising a particulate phase.

32. The cosmetic composition according to claim 31, wherein the particulate phase comprises particles chosen from pigments, pearlescent agents, and fillers.

33. The cosmetic composition according to claim 1, wherein it is in a form chosen from cast and compacted forms.

34. The cosmetic composition according to claim 1, wherein it is in the form of a lipstick.

35. A cosmetic method for the care and/or the making up of the skin, the lips and/or the superficial body growths, comprising applying to the skin, the lips and/or the superficial body growths a cosmetic composition comprising, in a physiologically acceptable medium, at least one fatty phase comprising
(i) at least one polyol ether chosen from polyalkylene glycol pentaerythritol ethers, present in an amount ranging from 0.5% to 40% by weight, relative to the total weight of the composition and
(ii) at least one pasty compound other than the at least one polyol ether, present in an amount ranging from 2% to 30% by weight, relative to the total weight of the composition.

36. The cosmetic method according to claim 35, wherein the cosmetic composition moisturizes the skin, the lips and/or the superficial body growths.

37. A cosmetic method for the care of chapped and/or dry lips, comprising applying to the lips a cosmetic composition comprising, in a physiologically acceptable medium, at least one fatty phase comprising
(i) at least one polyol ether chosen from polyalkylene glycol pentaerythritol ethers, present in an amount ranging from 0.5% to 40% by weight, relative to the total weight of the composition and
(ii) at least one pasty compound other than the at least one polyol ether, present in an amount ranging from 2% to 30% by weight, relative to the total weight of the composition.
